(19) United States
(12) Patent Application Publication
(10) Pub. No.: US 2016/0008241 A1
(43) Pub. Date: Jan. 14, 2016

(54) POWDERED COMPOSITION FOR USE IN THE PREPARATION OF WHITENING COSMETIC COMPOSITIONS

(71) Applicant: SENSIENT COSMETIC TECHNOLOGIES, Saint-Ouen l'Aumone (FR)

(72) Inventors: Enrico GALLINO, Suresnes (FR); Pamela EKLOU, Courbevoie (FR); Virginie BOULIER, Condecourt (FR); Marianne PELTIER, Villennes Sur Seine (FR)

(73) Assignee: SENSIENT COSMETIC TECHNOLOGIES, Saint-Ouen l'Aumone (FR)

(21) Appl. No.: 14/772,313

(22) PCT Filed: Mar. 3, 2014
(86) PCT No.: PCT/EP2014/054073

§ 371 Date: Sep. 2, 2015

(30) Foreign Application Priority Data
Mar. 5, 2013 (FR) 13 51935

(51) Int. Cl. A61K 8/29; A61K 8/02; A61Q 19/02; A61K 8/60; A61K 8/97; A61K 8/25; A61Q 1/02; A61K 8/86

(52) U.S. Cl. CPC A61K 8/29; A61Q 1/02; A61K 8/022; A61K 8/86; A61K 8/602; A61K 8/97; A61K 8/25; A61Q 19/02; A61K 2800/48; A61K 2800/596; A61K 2800/432; A61K 2800/26; A61K 2800/74

(57) ABSTRACT

The application relates to a powdered composition consisting in at least one pigment, at least one encapsulation agent, at least one whitening agent, at least one lightening agent, optionally an additive selected from vitamin B3, vitamin E, a ginseng extract, a red algae extract, a brown algae extract and mixtures thereof and optionally water, to its preparation method and to its use for preparing a cosmetic composition.

The present invention relates to a powdered composition for use in the preparation of cosmetic compositions having a whitening effect.

Many persons wish to have a paler, more radiant and/or more uniform complexion, and use cosmetic compositions for this purpose. Preferably, a whitening effect is then sought not only upon applying the cosmetic composition, but also during the time for which the whitening effect is produced in a longer term.

For this purpose, whitening and/or lightening agents have been developed and introduced into cosmetic compositions. Cosmetic compositions having a whitening or lightening effect are for example described in applications EP 1 859 835 (cosmetic composition for which the whitening agent is α-arbutin), CA 2,351,077 (cosmetic composition for which the whitening agent is ascorbic acid), FR 2 735 688 077 (cosmetic composition for which the whitening agent is an α-hydroxyacid in association with a titanium dioxide pigment) and FR 2 863 889 (cosmetic composition for which the whitening agent is an optical brightener).

Whitening agents are often not very stable since they degrade upon heat and/or by oxidation, notably in an aqueous medium. It is therefore necessary to develop cosmetic compositions or compositions for use in the preparation of cosmetic compositions, and for which the whitening agent is stabilized.

Moreover, the method for preparing a cosmetic composition typically comprises the mixing of different constituents of the cosmetic composition. This method is facilitated when <<all-in-one>> products comprising several of the constituents of the cosmetic composition may be used instead of the constituents taken individually. Thus, the method comprises less steps, involves less initial products and the sometimes expensive losses of constituents which may occur during the separate introduction of each constituent may be minimized.

Therefore there exists a need for providing <<all-in-one>>, products comprising a whitening agent for use in the preparation of cosmetic compositions having a whitening effect and:
for which the whitening effect is observable upon applying the cosmetic composition and is extended over time after its application, and
for which the whitening agent degrades less when it was introduced individually during the method for preparing the cosmetic composition.

### [Powdered Composition]

For this purpose, according to a first object, the invention relates to a powdered composition consisting in:
at least one pigment,
at least one encapsulant,
at least one whitening agent,
at least one lightening agent,
optionally an additive selected from vitamin B3, vitamin E, a ginseng extract, a red algae extract, a brown algae extract and mixtures thereof, and
optionally water.

This powdered composition is adapted so as to be introduced into a cosmetic composition. The inventors discovered that a cosmetic composition comprising this powdered composition has a whitening effect not only upon applying the cosmetic composition on the skin, but also an extended whitening effect over time when the cosmetic composition is applied daily, notably over a period of at least 9 days.

Without intending to be bound to particular theories, this extended effect over time would be due to the release of the whitening agent and/or of the lightening agent of the powdered composition, this release may notably occur during the preparation of the cosmetic composition, or upon application of the cosmetic composition.

Moreover, as explained above, the use of whitening agents is limited by the fact that these agents are often not very stable. Advantageously, the whitening agent and/or the lightening agent of the powdered composition degrade less than the same free whitening agents and/or lightening agents, because they are encapsulated in the encapsulant of the powdered composition. It is notably possible to store the powdered composition before it is introduced into a cosmetic composition without degradation of these agents.

The Pigment

The powdered composition comprises one or several pigments.

The pigment(s) present in the powdered composition according to the invention may independently be an organic, inorganic pigment or of a hybrid organic-inorganic nature or a mineral filler. These are typically inorganic pigments.

The inorganic pigment may for example be selected from titanium dioxide, zinc oxide, zirconium or cerium oxides, aluminium oxide, as well as iron or chromium oxides, manganese violet, ultramarine blue, chromium hydrate, ferric blue and mixtures thereof. The pigments may be used as such or optionally surface-treated, in the form of surface-treated pigments or of surface-treated nanopigments.

The preferred inorganic pigments are titanium dioxide, zinc oxide, zirconium dioxide, aluminium oxide and mixtures thereof, and preferably titanium dioxide or zinc oxide and mixtures thereof.

The organic pigment may for example be selected from carbon black, such as D&C black no. 2, red organic pigments, such as D&C Red no. 6, no. 7, no. 21, no. 27, no. 28, no. 30, no. 33, no. 36, no. 40, and cochineal carmine, blue organic pigments, such as copper phthalocyanine and FD&C Blue no. 1 lacquer, and yellow organic pigments such as D&C yellow no. 10 lacquer, FD&C yellow no. 5 and no. 6 lacquers.

As mineral fillers adapted to the application of the invention, mention may notably be made of talcum, kaolin, alumina or silica.

Generally, the composition comprises from 80% to 97% by weight of pigment(s) based on the total weight of the powdered composition.

Whitening Agent

The powdered composition according to the invention comprises one or several whitening agents.

A whitening agent is a synthetic or natural chemical compound which gives the possibility of having a depigmenting effect on skin. Two methods are customarily used for demonstrating this depigmenting effect:

either by quantifying the melanin level in epidermises, or by using the CIE Lab L*a*b* method for evaluating the color of the epidermises (the parameter L* indicating brightness and the parameters a* and b* indicating the sense of colors), for example by applying the whitening agents on pigmented human epidermises restored in vitro of the MelanoDerm® (MatTek) type. From these values, it is possible to calculate the color deviation (ΔE*) which characterizes the color difference between two points (ΔE* being all the higher than the skin is darker), calculated according to the formula:

ΔE*=\sqrt{(ΔL*)²+(Δa*)²+(Δb*)²}

These whitening agents may act according to several types of mechanisms:
by blocking the synthesis route of melanin at a given level (inhibition by enzymatic competition, blocking of active sites, suppression of synthesis or ripening of tyrosinase)
by blocking the transfer of melanosomas, and/or
by destroying melanocytes (a mechanism which is more difficult to control).
   Generally, the whitening agents act by a combination of several of these mechanisms.

The whitening agent(s) may independently be selected from kojic acid, α-arbutin, hydroquinone, vitamin C or one of its salts, ascorbyl glucoside, a licorice extract, azelaic acid, α-hydroxyacids (notably lactic acid or glycolic acid), aloesin, gallic acid and its derivatives (notably esters of gallic acid and of hydroflavanols, such as epicatechin-3-O-gallate (ECG), gallocatechin-3-O-gallate (GCG), epigallocatechin-3-O-gallate (EGCG), and epigallocatechin (EGG)), glutathione, glycolic acid, hexyl resorcinol, hydroxystilbene and its derivatives (notably resveratrol (3,4′,5-trihydroxystilbene) and its isomers (oxyresveratrol and methoxyresveratol)).

The preferred whitening agents are α-arbutin and liquorice extract. A liquorice extract is typically selected, for which the concentration of glycyrrhizic acid is greater than or equal to 0.15%, for example the one marketed by Alban Muller International under the trade name Botanical Liquorice PS.

Generally, the composition comprises from 1% to 5% by weight of whitening agent(s) based on the total weight of the powdered composition.

Lightening Agent

The powdered composition according to the invention comprises one or several lightening agents.

A lightening agent gives the possibility of intensifying the radiance and sharpening the hues of the cosmetic compositions comprising them upon application on the skin.

The lightening effect may notably be measured with a chromatometer (colorimeter), in particular by using the CIE Lab L*a*b* method, for example by applying lightening agents on pigmented human epidermises restored in vitro of the MelanoDerm® (MatTek) type. From these values, it is possible to calculate the color (ΔE*) as explained above.

The lightening agent(s) may independently be selected from optical brighteners and mothers-of-pearls (such as borosilicates or natural or synthetic mica (such as synthetic fluorphlogopite), and, said mica being optionally covered with titanium oxide (TiO₂), iron oxide (Fe₂O₃) (red, yellow or black), chromium oxide (Cr₂O₃), carmine, Prussian blue, ultramarine blue, manganese violet or with their mixtures), silica, alumina and mixtures thereof).

Optical brighteners are lightening agents via an optical route, and are chemical compounds provided with fluorescence properties, which absorb in the ultraviolet (maximum absorption at a wavelength of less than 400 nm) and re-emit energy by fluorescence in the wavelength range comprised between 380 nm and 830 nm. Emission of energy comprised between 400 nm and 480 nm results in an emission in the blue portion of the visible range, which contributes, when this emission occurs on the skin, to visually whitening it.

From among optical brighteners, mention may be made of:
1) bis(benzoxazol-2-yl) of formula (I):

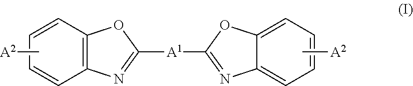

wherein A¹ is an aromatic, heterocyclic or alkyl group, and A² is a hydrogen atom or an alkylene group;
2) cumarins of formula (II):

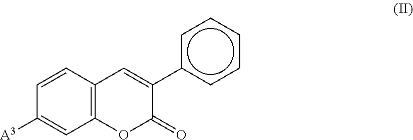

wherein A³ is a heterocyclic group;
3) bis(styryl)biphenyl of formula (III)

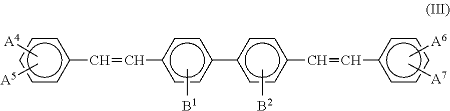

wherein A⁴, A⁵, A⁶, A⁷, B¹ and B² represent independently a hydrogen atom, a group —SO₃Na or an alkyl group; and
4) derivatives of triazine-stilbene of formula (IV):

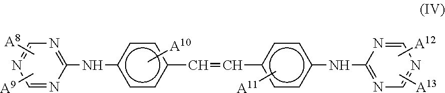

wherein A⁸, A⁹, A¹² and A¹³ represent independently a hydrogen atom, a group —SO₃Na or a phenylamino, dialkylamino or morpholino group, and A¹⁰ and A¹¹ represent independently a hydrogen atom or a -SO₃Na group.

In the formulae (I) to (IV):
the aromatic groups used may be groups including one or several benzene rings, for example phenyl, biphenyl, substituted phenyl groups or aromatic groups formed from polycyclic aromatic hydrocarbons, such as naphthyl, phenanthryl, anthracenyl, fluoranthenyl groups.
the heterocyclic groups are saturated, unsaturated or aromatic cyclic groups including carbon atoms, notably from 3 to 10, and one or several heteroatoms (notably 1, 2 or 3) such as O, N and S. As an example of heterocyclic groups, mention may be made of thienyl, furyl, pyrannyl, isobenzofuranyl, isobenzothienyl, pyrrolyl, pyridyl, pyrazolyl groups.
the alkyl groups are linear or branched monovalent hydrocarbon groups preferably having from 1 to 16 carbon atoms, for example the tert-butyl group.
the alkylene groups are linear or branched divalent hydrocarbon groups, preferably having from 1 to 16 carbon atoms, for example methylene or ethylene.

The preferred optical brighteners are sodium 4,4′-bis[(4,6-dianilino-1,3,5-triazin-2-yl)amino]stilbene2,2′-disulfonate, 2,5-thiophenedi-ylbis(5-terbutyl-1,3-benzoxazole), sodium di-styryl-20 4,4′-biphenyldisulfonate, and disodium distyrylbiphenyl disulfonate.

Preferably, the lightening agent is natural or synthetic mica (such as synthetic fluorphlogopite), and optionally covered with titanium oxide (TiO₂), iron oxide (Fe₂O₃) (red, yellow or black), chromium oxide (Cr₂O₃), carmine, Prussian blue, ultramarine blue, manganese violet, silica, alumina or mixtures thereof.

Generally, the composition comprises from 1% to 15% by weight of lightening agent(s) based on the total weight of the powdered composition.

Encapsulant

The powdered composition according to the invention comprises one or several encapsulants.

The encapsulant typically has a melting point comprised between 40° and 50° C.

Generally, the encapsulant is transparent, in order not to modify the white color of the pigment of the powdered composition.

Preferably, an encapsulant is selected for which the viscosity is from 10 cSt to 800 cSt (0.1 cm².s⁻¹ to 8 cm².s⁻¹), notably 50 cSt to 200 cSt (0.5 cm².s⁻¹ to 2 cm².s⁻¹) at the temperature for preparing the powdered composition (temperature of step a) below) is adapted so that it may easily be introduced into the powdered composition.

The encapsulants may be hydrophilic or lipophilic. A hydrophilic encapsulant will be preferred if the cosmetic composition into which it is desired to introduce the powdered composition is aqueous or is an emulsion. A lipophilic encapsulant will be preferred if the powdered composition into which it is desired to introduce the cosmetic composition is oily or is an emulsion.

Among the hydrophilic encapsulants which may be used, mention may be made of film-forming agents of natural origin from the family of polysaccharides (as an example: alginates, pullulan, sodium hyaluronate, chitin, dextran, carrageenan, chitosan succinamide, galactomannans), esters of waxes and of polyglycerol, or synthetic polymers such as polyethylene glycol, polyacrylic acid, polylactic acid, polyvinyl alcohol, acrylamide, isopropylacrylamide or copolymers thereof.

Among the lipophilic encapsulants which may be used, mention may be made of natural waxes of vegetable origin (for example candelila, carnauba waxes), of animal origin (for example beeswax), of mineral origin or synthetic origin (for example: polyethylene, polypropylene, . . . ).

Are preferred polyethylene glycol, a copolymer of polyethylene glycol, or a natural or synthetic wax. Polyethylene glycol, notably with a molecular mass from 500 to 9,000g.mol⁻¹, in particular between 3,000 and 4,000g.mol⁻¹, is a more preferred encapsulant.

Generally, the composition comprises from 1% to 5% by weight of encapsulant based on the total weight of the powdered composition.

Other Additive

The powdered composition may comprise an additive selected from vitamin B3, vitamin E, a ginseng extract, a red and brown algae extract and mixtures thereof.

These additives have the advantage of giving additional properties to the powdered composition, and therefore to the cosmetic composition into which it will be introduced:
vitamin B3 gives the possibility of controlling the color of the powdered composition,
vitamin E has antioxidant properties,
ginseng extract stimulates the production of collagen, has a repairing and healing effect on the skin and protects the skin against premature ageing,
the red algae extract protects the skin against premature ageing,
the brown algae extract has gelling, stabilizing and thickening properties.

Generally, the composition comprises from 0% to 5% by weight of said additive based on the total weight of the powdered composition.

Water

The powdered composition may comprise water, notably when the encapsulant is hydrophilic.

The water proportion has to be sufficiently small so that the composition remains in a powdered form. Generally, the composition comprises from 0% to 5% by weight of water based on the total weight of the powdered composition.

Proportions of the Components

According to an embodiment, the powdered composition consists of:
80 to 97% by mass of pigment(s), and/or
1 to 5% by mass of encapsulant(s), and/or
1 to 5% by mass of whitening agent(s), and/or
1 to 15% by mass of lightening agent(s), and/or
0 to 5% by mass of additive(s) selected from vitamin B3, vitamin E, a ginseng extract, a red and brown algae extract and mixtures thereof, and/or
0 to 5% by mass of water.

### [Method for Preparing the Powdered Composition]

According to a second object, the invention relates to the method for preparing the powdered composition as defined above, comprising the step for mixing at least one pigment, at least one encapsulant, at least one whitening agent, at least one lightening agent, optionally one additive as defined above and optionally water.

The mixing of the components of the powdered composition may be applied by simultaneous or separate mixing of the components.

Typically, the method comprises the steps:
a) mixing at least one encapsulant and at least one whitening agent, preferably at a temperature from room temperature (about 20° C.) to a temperature less than the degradation temperature of the whitening agent(s),
b) mixing at least one pigment and at least one lightening agent, preferably at a temperature from room temperature (about 20° C.) to a temperature of less than the degradation temperature of the lightening agent(s) and the degradation temperature of the pigment(s),
c) dispersing the mixture obtained in step a) on the mixture obtained in step s b), preferably at a temperature from room temperature (about 20° C.) to a temperature of less than the degradation temperature of each of the components (whitening agent, lightening agent, additive, pigment).

When the encapsulant is hydrophilic and the powdered composition comprises water, the water is generally mixed with the encapsulant and the whitening agent during step a).

Preferably, no organic solvent is used in the preparation method according to the invention.

The pigments, whitening agents, lightening agents, encapsulants and additives are generally commercially available.

The invention also relates to the powdered compositions which may be obtained with this method.

### [Cosmetic Use of the Powdered Composition]

According to a third object, the invention relates to the use of the powdered composition for introduction into a cosmetic composition (or for preparing a cosmetic composition), as well as to a cosmetic composition comprising the powdered composition.

The powdered composition according to the invention corresponds to an “all-in-one product” adapted so as to be introduced into a cosmetic composition. The use of the powdered composition is economically advantageous since it is possible to introduce in a single step, the pigment, the whitening agent, the lightening agent and the encapsulant, which leads to a gain in time and may minimize the losses of compounds during production.

The cosmetic composition typically comprises the powdered composition as defined above and at least one cosmetically acceptable excipient. The proportion by mass of powdered composition based on the weight of the cosmetic composition is for example from 1 to 3%.

The cosmetic composition may appear as a monophasic or biphasic lotion, water-in-oil or oil-in-water emulsion, hydrophilic or lipophilic gel or powder.

The cosmetic composition may for example be a hair care product (a hair coloration product, shampoo, conditioner . . . ), a makeup product (a mascara, a foundation, an eye-liner, an eye shadow or a blush, a lipstick, a lip gloss, a nail varnish) or a face or body care product. Preferably, this is a makeup or care product for topical application.

The invention is illustrated by the following examples.

### EXAMPLES

### Example 1

### Preparation of a Powdered Composition According to the Invention

1 kg of distilled water and 2 kg of polyethylene glycol (Carbowax Sentry PEG 1450—Dow Chemicals) (encapsulant) were introduced into a beaker and mixed at room temperature until a limpid solution is obtained. According to an alternative method, the mixture of polyethylene glycol/water was heated to 50° C. until a limpid solution was obtained.

0.6 kg of α-arbutin (DSM Pentapharm) (whitening agent) and 1 kg of liquorice extract (Botanical-Licorice PS—Alban Muller International) (whitening agent) were added and the mixture was stirred until a homogenous solution was obtained.

In a mixer, were introduced 50 kg of titanium oxide (Sensient Cosmetic Technologies) (pigment) and 6 kg of mica (Sensient Cosmetic Technologies) (lightening agent) which were placed with stirring at 45° C. When the mixture of powders reached 45° C., the prepared mixture of distilled water/polyethylene glycol/α-arbutin/licorice extract was added thereto dropwise, and then the mixture was left to homogenize for 3 hours at 45° C.

60 kg of powdered composition were thus prepared.

### Example 2

### The Use of the Powdered Composition According to Example 1 for Preparing a Cosmetic Composition

The powdered composition of Example 1 was introduced into two cosmetic compositions with a proportion of 3% by weight based on the weight of the cosmetic composition. The latter had the composition specified in the following Tables 1 and 2 and were prepared as described hereafter.

TABLE 1
Composition of the cosmetic composition 1

| | | | | Percentage |
|---|---|---|---|---|
| Phase | Product | INCI | Supplier | (%) |
| Phase | Emulsiphos | Potassium Cethyl | Symrise | 2 |
| A | | Phosphate and | | |
| | | Hydrogenated Palm | | |
| | | Glycerides | | |
| | Eutanol G | Octyldodecanol | Basf | 2 |
| | Miglyol 812 | Caprylic/capryl | Sasol | 8 |
| | | Triglyceride | | |
| | DC FZ 3196 | Caprylyl Methicone | Dow Corning | 2 |
| | Cetiol C5 | Coco-Caprylate | Basf | 1 |
| Phase | Water | Aqua | — | 73.4 |
| B | Glycerine | Glycerin | Alban Muller | 3 |
| | | | International | |
| | Microcare PM3 | Phenoxyethanol and | Thor | 0.5 |
| | | Methyl Paraben and | | |
| | | Ethyl Paraben and | | |
| | | Propyl Paraben | | |
| Phase | Thickagent LC | Xanthan gum and | Sensient | 1 |
| C | | Hectorite and | | |
| | | Cellulose | | |
| Phase | Hydrofacteur LC WP | Lactic acid and Urea | Sensient | 2 |
| D | | and Sorbitol and | | |
| | | Hydrolyzed wheat | | |
| | | protein and Glycerin | | |
| | | and Allantoin and | | |
| | | Aqua and Butylene | | |
| | | glycol and | | |
| | | Triethanolamine | | |
| Phase | Powdered composition | — | Sensient | 3 |
| E | according to example 1 | | | |
| | Covabead PMMA 2 MUSI | Methyl methacrylate | Sensient | 2 |
| | | crosspolymer and | | |
| | | silica | | |
| Phase | Procare perfume | — | Sensient | 0.1 |
| F | | | | |

The cosmetic preparation 1 was prepared according to the following procedure:
Heat phase A to 45° C.
Add phase C to phase B with stirring until homogenization and then heat to 85° C.
Add phase A to phases B and C
At room temperature, add phase D to the mixture of phases A+B+C
Add phase E to the mixture of phases A+B+C+D
Add phase F to the mixture of phases A+B+C+D+E

TABLE 2
Composition of the cosmetic composition 2

| | | | | Percentage |
|---|---|---|---|---|
| Phase | Product | INCI | Supplier | (%) |
| Phase | Dracorin CE | Glyceryl | Symrise | 3 |
| A | | Stearate Citrate | | |
| | Miglyol 812 | Caprylic/capryl | Sasol | 8 |
| | | Triglyceride | | |
| | Isononyl | Isononyl | Mosselman | 2.5 |
| | Isononanoate | Isononanoate | | |
| | Ecorol 68/50P | Cetyl stearyl | YDS | 1.5 |
| | | alcohol | Chemical | |
| | DC FZ 3196 | Caprylyl | Dow Corning | 2 |
| | | Methicone | | |
| | DC 200, 50 cst | Dimethicone | Dow Corning | 2 |
| Phase | Water | Water | — | 67.9 |
| B | Glycerine | Glycerin | Alban Muller | 3 |
| | | | International | |
| | Butylene glycol | Butylene glycol | Kyowa | 2 |
| | | | hakko | |
| | | | europe | |
| | Fucosorb WP | Algae and | Sensient | 2 |
| | | Sorbitol and | | |
| | | aqua | | |
| | Microcare PM3 | Phenoxyethanol | Thor | 0.5 |
| | | and Methyl | | |
| | | Paraben and | | |
| | | Ethyl Paraben | | |
| | | and Propyl | | |
| | | Paraben | | |
| Phase | Covacryl RH | Sodium | Sensient | 0.5 |
| C | | polyacrylate | | |
| Phase | Powdered | | Sensient | 3 |
| D | composition | | | |
| | according to | | | |
| | Example 1 | | | |
| | Submica FL | Mica | Sensient | 2 |
| Phase | Procare perfume | | Sensient | 0.1 |
| E | | | | |

The cosmetic preparation 2 was prepared according to the following procedure:
Heat phase A to 45° C.
Add phase C to phase B with stirring until homogenization and then heat to 85° C.
Add phase A to the phases B and C
At room temperature, add phase D to the mixture of phases A+B+C
Add phase E to the mixture of phases A+B+C+D

The whitening effect of the cosmetic composition was demonstrated by carrying out tests in vitro of its depigmenting potential on a human epidermis model over a period of 9 days. The method was based on the measurement of the melanin content and on the coloration of the tissues. The investigation was carried out on pigmented human epidermises restored in vitro of the MelanoDerm™ type, provided by MatTek. This model was obtained by co-cultures of human melanocytes and keratinocytes from Afro-American donors, sown, in order to obtain a 3D epidermal tissue with features (differentiation and pigmentation) close to human skin.

The cosmetic composition containing the powdered composition was applied in an amount of 4-5 mg/cm² directly on the surface of the epidemises by means of an applicator. After each application, the surface of the tissues was rinsed with a phosphate buffer solution. The tissues were then put back to incubate at 37° C., in an air-CO₂ oven. The test formula was applied at D0, D1, D2, D5, D6, D7, D8. The melanin content and the coloration of the tissues were analyzed at the end of the treatment (D9). The results obtained with the cosmetic composition containing the powdered composition was compared with those obtained with:
a non-treated epidermis (control)
an epidermis treated with a commercial depigmenting cream (Melanex® Trio, marketed by Laboratoire Expanscience).

The coloration of the epidermises was measured at DO (before applying the test element) and 24 hours after the last application by means of a chromatometer. In particular, the parameters L*, which indicate the brightness, and a* and b*, which indicate the color sense, were measured. From these values, it was possible to calculate:
1) The individual typological angle)(ITA° calculated according to the formula:

ITA°=[Atan (L*−50)/b*]×180/π
This parameter defines the pigmentation degree of the skin (the lower the ITA°, the more the skin is pigmented)
2) The color deviation (ΔE*) which characterized the color difference between two points (ΔE* is all the higher since the skin is dark), calculated according to the formula:

ΔE*=\sqrt{(ΔL*)²+(Δa*)²+(Δb*)²}

In order to evaluate the melanin content, at the end of the tests, the tissues were heated for 45-60 minutes to 100° C. in a solubilizing base (Solvable™). After cooling the extracts and centrifugation, the absorbance of the solutions was read out at 405 nm by means of a microplate reader. A standard range consisting of synthetic melanin was incubated at 100° C. for the same period of time as that of the cell extracts, which allows transformation of the optical densities (O.D₄₀₅) into melanin units (μg/ml).

The results of coloration of the epidermis (ΔITA° and ΔE*) and of depigmenting activity (D.A. %) are summarized in the following table:

| | | | | Tissue |
|---|---|---|---|---|
| | ΔITA° = (ITA° | | D.A. | viability |
| Samples | D9 − ITA° D0) | ΔE* | (%) | (%) |
| Control | −71.95 | 22.88 | — | 100 |
| Melanex ® Trio | −26.76 | 14.71 | 57% | 48 |
| (comparative) | | | | |
| Cosmetic | −46.01 | 19.60 | 23% | 76 |
| composition | | | | |
| containing the | | | | |
| powdered | | | | |
| composition | | | | |

The depigmenting activity was calculated according to the formula:

D.A. %=[(Melanin_{control}−Melanin_{treated})/Melanin_{control}]*100

wherein Melanin_{control} and Melanin_{treated} respectively represent the melanin concentrations in the control sample and in the sample treated with a whitening cosmetic composition. These results demonstrate that the application of different cosmetic compositions lead to a decrease in the coloration of the tissues, to whitening of the skin as well as to a reduction in the melanin content.

The powdered composition of Example 1 also gives the possibility of obtaining better tissue viability as compared with a commercial whitening agent. Cutaneous acceptability was evaluated by a MTT test. For both cosmetic preparations containing the powdered composition and the ingredients introduced separately, the depigmenting effect is not related to a cytotoxic effect since the viability of the tissues is not modified. On the other hand, the Melanex Trio preparation induces slight lowering (25%) of the MTT response of the tissues.

1. A powdered composition comprising in:
at least one pigment,
at least one encapsulant,
at least one whitening agent,
at least one lightening agent,
optionally an additive selected from vitamin B3, vitamin E, a ginseng extract, a red algae extract, a brown algae extract and mixtures thereof, and
optionally water.

2. The powdered composition according to claim 1, wherein each pigment is independently selected from an organic pigment, an inorganic pigment, a pigment of an organic-inorganic hybrid nature and a mineral filler.

3. The powdered composition according to claim 2, wherein each pigment is independently selected from:
an inorganic pigment selected from titanium dioxide, zinc oxide, zirconium or cerium oxides, aluminium oxide, as iron or chromium oxides, manganese violet, ultramarine blue, chromium hydrate and ferric blue,
an organic pigment selected from carbon black, red organic pigments, cochineal carmine, blue organic pigments, and yellow organic pigments and
a mineral filler selected from talcum, kaolin, alumina and silica.

4. The powdered composition according to claim 1, wherein each whitening agent is independently selected from kojic acid, α-arbutin, hydroquinone, vitamin C, a salt of vitamin C, ascorbyl glucoside, licorice extract, azelaic acid, an α-hydroxyacid, aloesine, gallic acid, a derivative of gallic acid, glutathione, glycolic acid, hexyl resorcinol, hydroxystilbene and a derivative of hydroxystillbene.

5. The powdered composition according to claim 1, wherein each lightening agent is independently selected from optical brighteners and mothers-of-pearl.

6. The powdered composition according to claim 1, wherein each encapsulant is independently selected from:
a hydrophilic encapsulant selected from film-forming agents of natural origin from the family of polysaccharides, esters of waxes and polyglycerol, and synthetic polymers and
a lipophilic encapsulant selected from natural waxes of vegetable, animal, mineral and synthetic origin.

7. The powdered composition according to claim 1, wherein:
the pigment is selected from titanium dioxide, zinc oxide and mixtures thereof, and/or
the whitening agent is selected from α-arbutin and liquorice extract, and/or
the lightening agent is mica optionally covered with titanium oxide, red, yellow or black iron oxide, chromium oxide, carmine, Prussian blue, ultramarine blue, manganese violet, silica, alumina and mixtures thereof, and/or
the encapsulant is a polyethylene glycol.

8. The powdered composition according to claim 1, comprising:
80 to 97% by mass of pigment(s), and
1 to 5% by mass of encapsulant(s), and
1 to 5% by mass of whitening agent(s), and
1 to 15% by mass of lightening agent(s), and
0 to 5% by mass of additive(s) selected from vitamin B3, vitamin E, a ginseng extract, a red and brown algae extract and mixtures thereof, and
0 to 5% by mass of water.

9. A method for preparing a powdered composition according to claim 1, comprising the step of mixing at least one pigment, at least one encapsulant, at least one whitening agent, at least one lightening agent, optionally an additive selected from vitamin B3, vitamin E, a ginseng extract, a red algae extract, a brown algae extract and mixtures thereof, and optionally water.

10. (canceled)

11. The powdered composition according to claim 3, wherein the pigment is selected from lacquers of D&C black no. 2, D&C Red no. 6, D&C Red no. 7, D&C Red no. 21, D&C Red no. 27, D&C Red no. 28, D&C Red no. 30, D&C Red no. 33, D&C Red no. 36, D&C Red no. 40, copper phthalocyanine, FD&C blue no. 1 lacquer, D&C yellow no. 10, FD&C yellow no. 5 and FD&C yellow no. 6.

12. The powdered composition according to claim 5, wherein the lightening agent is selected from borosilicates and natural or synthetic mica, said mica being optionally covered with titanium oxide, red, yellow or black iron oxide, chromium oxide, carmine, Prussian blue, ultramarine blue, manganese violet, silica, alumina or mixtures thereof.

13. The powdered composition according to claim 6, wherein the synthetic polymer is selected from polyethylene glycol, polyacrylic acid, polylactic acid, polyvinylalcohol, acrylamide, isopropylacrylamide and copolymers thereof.

14. The powdered composition according to claim 6, wherein each encapsulant is independently selected from a polyethylene glycol, a copolymer of polyethylene glycol, a natural wax, and a synthetic wax.
